(19) United States
(12) Patent Application Publication
(10) Pub. No.: US 2024/0198946 A1
(43) Pub. Date: Jun. 20, 2024

(54) SAFE DRIVING DETERMINATION APPARATUS

(71) Applicant: ISUZU MOTORS LIMITED, Tokyo (JP)

(72) Inventor: Kazuhiko YAMAGUCHI, Fujisawa-shi, Kanagawa (JP)

(21) Appl. No.: 17/908,901

(22) PCT Filed: Mar. 9, 2021
(86) PCT No.: PCT/JP2021/009366

§ 371 Date: Sep. 1, 2022

(30) Foreign Application Priority Data
Mar. 11, 2020 (JP) 2020-042076

(51) Int. Cl. B60R 21/015; G06T 7/70; G06V 20/59

(52) U.S. Cl. CPC B60R 21/01566; G06T 7/70; G06V 20/597; G06T 2207/30201; G06T 2207/30268

(57) ABSTRACT

A safe driving determination apparatus includes a state identification part that identifies a state of the vehicle, an angle value identification part that identifies an angle value indicating a face direction angle of a driver with respect to the traveling direction, and a determination part that determines whether or not the driver is in a state of being inattentive to the road ahead, wherein the angle value calculation part inputs the angle value calculated to the determination part if the state is in a state where the driver is not allowed to be in a state of being inattentive to the road ahead, and inputs a value at which the difference from the reference angle value is less than the threshold value, to the determination part if the state is in a state where the driver is allowed to be in a state of being inattentive to the road ahead.

### TECHNICAL FIELD

The present disclosure relates to a safe driving determination apparatus.

### BACKGROUND ART

Conventionally, an apparatus has been proposed that determines whether a driver is inattentive to the road ahead (looking aside) according to a face direction of a driver while driving a vehicle, and alerts the driver if the driver is determined to be inattentive to the road ahead. Patent document 1 discloses a device for determining a state of being inattentive that has a function of making not to determine that the driver is inattentive to the road ahead so easily if a vehicle is traveling on a curved road and a driver's face is facing the destination of the curved road.

### PRIOR ART

### Patent Document

Patent Document 1: Japanese Unexamined Patent Application Publication No. 2019-70876

### SUMMARY OF INVENTION

### Problems to be Solved by the Invention

A conventional device for determining a state of being inattentive determines whether or not the driver is in a state of being inattentive to the road ahead on the basis of whether or not an angle value indicating a driver's face direction is greater than a reference value. If the conventional device for determining a state of being inattentive detects that the vehicle is traveling on a curved road, the device uses a reference value for the curved road that is different from the reference value used while the vehicle is traveling on a road other than a curved road, making not to determine that the driver is in a state of being inattentive to the road ahead on the curved road easily.

However, the angle value of the face direction on the curved road varies depending on the driver. Therefore, even if the reference value for the curved road is used, the device for determining a state of being inattentive may erroneously determine that the driver is in a state of being inattentive to the road ahead despite the fact that the vehicle is on the curved road.

The present disclosure focuses on this point, and an object thereof is to prevent or reduce an erroneous determination that a driver is inattentive to the road ahead in a state where the driver does not need to be attentive to the road ahead.

### Means for Solving the Problems

A safe driving determination apparatus according to an embodiment of the present disclosure is a safe driving determination apparatus for acquiring an angle value indicating a face direction angle of a driver by using a traveling direction of a vehicle as a reference after capturing an image of the driver of the vehicle with an imaging device to determine whether or not the driver is in a state of being inattentive to the road ahead on the basis of the acquired angle value, the apparatus includes a state identification part that identifies a state of the vehicle, an angle value identification part that identifies an angle value indicating a face direction angle of at least one of i) a face direction angle of the driver in a yaw direction with respect to the traveling direction or ii) a face direction angle of the driver in a pitch direction with respect to the traveling direction, and a determination part that determines whether or not the driver is in a state of being inattentive to the road ahead if a difference between the angle value and a reference angle value is equal to or greater than a threshold value, wherein the angle value calculation part inputs the angle value calculated to the determination part if the state identified by the state identification part is in a state where the driver is not allowed to be in a state of being inattentive to the road ahead, and inputs a value at which the difference from the reference angle value is less than the threshold value, as the angle value to the determination part if the state identified by the state identification part is in a state where the driver is allowed to be in a state of being inattentive to the road ahead.

The state identification part may identify a state of the vehicle as a state where the driver is allowed to be in a state of being inattentive to the road ahead if the vehicle is stopped or if the vehicle is decelerating and the speed of the vehicle is equal to or lower than a threshold speed.

The state identification part may identify a state of the vehicle as a state where the driver is allowed to be in a state of being inattentive to the road ahead if the vehicle is putting on a blinker or if the vehicle is traveling on a curved road.

The state identification part may identify that a state of the vehicle is a state where the driver is not allowed to be in a state of being inattentive to the road ahead if a blinker is on even after a threshold time has elapsed since the vehicle starts to putting on a blinker.

The determination part may determine that the driver is in a state of being inattentive to the road ahead if a difference between the angle value and the reference angle value is equal to or greater than a threshold value, and may determine that the driver is not in a state of being inattentive to the road ahead if a difference between the angle value and the reference angle value is less than the threshold value.

### Effect of the Invention

According to the present disclosure, it is possible to prevent or reduce an erroneous determination that a driver is inattentive to the road ahead in a state where the driver does not need to be attentive to the road ahead.

### BRIEF DESCRIPTION OF THE DRAWINGS

FIG. 1 shows a configuration of a vehicle S according to the present disclosure.

FIG. 2 shows a relationship between an imaging device 1 and a driver's face direction angle.

FIG. 3 is a flowchart showing a process in which an angle value calculation part 422 determines an angle value to be notified to a determination part 423.

### DESCRIPTION OF EMBODIMENTS

### <Configuration of Vehicle S>

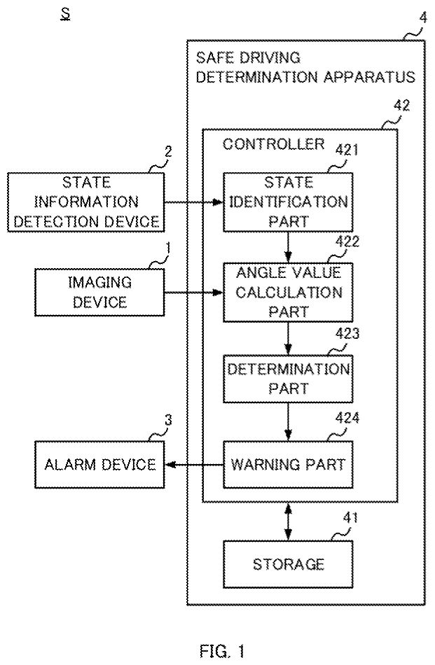

FIG. 1 shows a configuration of a vehicle S according to the present disclosure. The vehicle S includes an imaging device 1, a state information detection device 2, an alarm device 3, and a safe driving determination apparatus 4.

The imaging device 1 is provided to a driver's seat of the vehicle S, and includes a CCD camera, for example. The imaging device 1 captures an image of a driver sitting in the driver's seat from the front to generate a captured image. For example, the imaging device 1 captures an image of a driver's face while the vehicle S is traveling, and generates the captured image that enables identification of the driver's face direction angle with respect to the traveling direction of the vehicle S. The imaging device 1 outputs the generated captured image to the safe driving determination apparatus 4.

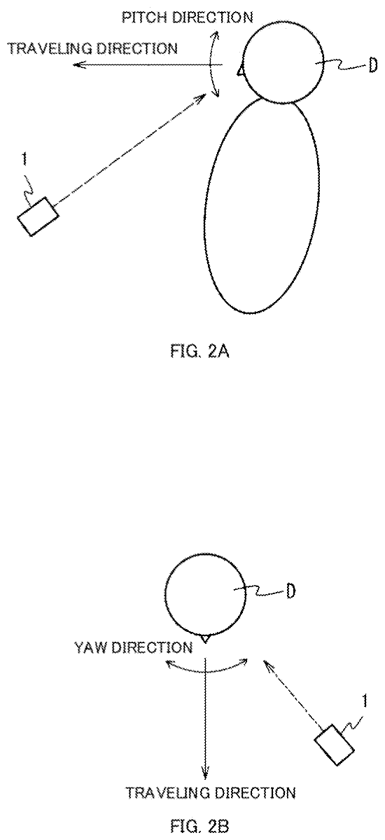

FIG. 2 shows a relationship between the imaging device 1 and the driver's face direction angle. FIG. 2(a) shows a side view of a driver D, and FIG. 2(b) shows a top view of the driver D. As shown in FIGS. 2(a) and 2(b), the imaging device 1 is not in front of a driver D's face, but is provided diagonally downward from the driver D's face, for example.

The state information detection device 2 detects state information necessary for identifying a state of the vehicle S traveling. The state of the traveling vehicle S includes stopping, decelerating, putting on a blinker, and traveling on a curved road, for example. The state information is information on the speed, acceleration, blinker operation, and the road on which the vehicle S is traveling. The state information detection device 2 notifies the safe driving determination apparatus 4 about detected state information.

The state information detection device 2 includes a CCD camera, for example, and transmits a captured image of the road in front of the vehicle S during traveling, to the safe driving determination apparatus 4. The state information detection device 2 is connected to a blinker of the vehicle S, and determines whether or not the blinker is operating and notifies the safe driving determination apparatus 4 about the determination, for example. The state information detection device 2 notifies the safe driving determination device 4 about a detected speed of the vehicle S, for example.

The alarm device 3 is a device that issues a warning to the driver on the basis of notification from the safe driving determination apparatus 4 that the driver is inattentive to the road ahead (for example, looking aside) while driving. The alarm device 3 includes a speaker for issuing an alarm, a display part for displaying a warning screen, and a vibration generation part for generating vibration, for example. It should be noted that the alarm device 3 may issue the warning by combining at least two of sound, display, and vibration.

The safe driving determination apparatus 4 includes a storage 41 and a controller 42. The safe driving determination apparatus 4 captures the image of the driver of the vehicle S using the imaging device 1 to identify an angle value indicating the driver's face direction angle when referenced to the traveling direction of the vehicle. The safe driving determination apparatus 4 determines whether or not the driver is in a state of being inattentive to the road ahead on the basis of the acquired angle value. If the safe driving determination apparatus 4 determines that the driver is in the state of being inattentive to the road ahead, the safe driving determination apparatus 4 notifies the alarm device 3 to issue the warning.

The storage 41 includes a storage medium such as a Read Only Memory (ROM), a Random Access Memory (RAM), and a hard disk. The storage 41 stores a program executed by the controller 42, which will be described later. The storage 41 stores information on the driver's face direction angle and information on the state of the vehicle S, for example.

The controller 42 is a Central Processing Unit (CPU), for example. The controller 42 functions as a state identification part 421, an angle value calculation part 422, a determination part 423, and a warning part 424 by executing the program stored in the storage 41. The controller 42 identifies the driver's face direction angle and the state of the vehicle S, and determines whether or not the driver is in the state of being inattentive to the road ahead.

### <Operation of State Identification Part 421>

The state identification part 421 acquires, from the state information detection device 2, a captured image of the road in front of the vehicle S, information on whether or not the blinker of the vehicle S is operating, and the state information indicating the speed of the vehicle S. The state identification part 421 determines whether or not the driver is allowed to be in the state of being inattentive to the road ahead on the basis of the acquired state information. The state identification part 421 notifies the angle value calculation part 422 about a determination result.

The state identification part 421 determines whether or not the road on which the vehicle S is traveling is a curved road, on the basis of the captured image of the road in front of the vehicle S acquired from the state information detection device 2, for example. If the state identification part 421 determines that the vehicle S is traveling on a curved road, the state identification part 421 identifies that the state of the vehicle S is a state where the driver is allowed to be in the state of being inattentive to the road ahead.

Further, the state identification part 421 acquires the state information indicating whether or not the blinker is operating, from the state information detection apparatus 2. If the state identification part 421 determines that the vehicle S is putting on the blinker on the basis of the acquired state information, the state identification part 421 identifies that the state of the vehicle S is a state in which the driver is allowed to be in the state of being inattentive to the road ahead.

However, the state identification part 421 identifies that the state of the vehicle S is a state where the driver is not allowed to be in the state of being inattentive to the road ahead if the blinker of the vehicle S is still on even after a threshold time has elapsed after the vehicle S starts putting on the blinker. The threshold time is a maximum value of a time required from when the vehicle S starts putting on blinker to when the blinker is off, and is a maximum value of a time required for the vehicle S to change a course or turn left or right, for example. By having the state identification part 421 operate in this manner, the safe driving determination apparatus 4 can determine whether or not the driver is in the state of being inattentive to the road ahead using the determination part 423, which will be described later if, for example, the driver fails to off the blinker.

The state identification part 421 acquires the speed of the vehicle S from the state information detection device 2. The state identification part 421 may determine whether or not the vehicle S is stopping, accelerating, and decelerating on the basis of the acquired speed of the vehicle S. The state identification part 421 identifies that the state of the vehicle S is a state where the driver is allowed to be in the state of being inattentive to the road ahead, if the vehicle S is stopped or if the vehicle S is decelerating and the speed of the vehicle is equal to or lower than a threshold speed. The threshold speed is a maximum value of the speed at which the driver can check left and right for turning the vehicle S to the left and right, for example. In this way, the state identification part 421 identifies that the driver is allowed to be inattentive to the road ahead if the speed of the vehicle S is below the threshold speed, such that the safe driving determination apparatus 4 can be restricted from erroneously determining, for example, the state where the driver is checking left and right to make a left or right turn as that the driver is in the state of being inattentive to the road ahead.

### <Operation of the Angle Value Calculation Part 422>

The angle value calculation part 422 calculates the angle value on the basis of the captured image captured by the imaging device 1. The angle value calculation part 422 calculates the angle value indicating at least one of i) the driver's face direction angle in the yaw direction with respect to the traveling direction (FIG. 2(b)) or ii) the driver's face direction angle in the pitch direction with respect to the traveling direction (FIG. 2(a)).

The angle value calculation part 422 receives a notification of the determination result of whether or not the driver is allowed to be in the state of being inattentive to the road ahead, from the state identification part 421. The angle value calculation part 422 determines the angle value to be notified to the determination part 423 on the basis of the determination result notified from the state identification part 421. Specifically, if the state identification part 421 determines that the driver is allowed to be in the state of being inattentive to the road ahead, the angle value calculation part 422 corrects the calculated angle value to a value at which an angle difference, which is the difference between the calculated angle value and the reference angle value, is less than the threshold value. The threshold value is the minimum value of the angle difference for the determination part 423 to determine that the driver is in the state of being inattentive to the road ahead.

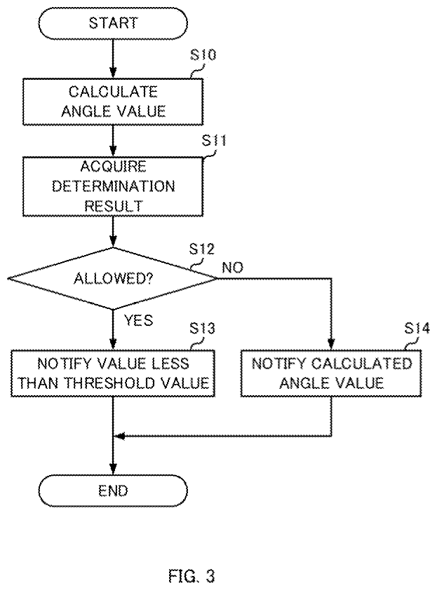

FIG. 3 is a flowchart showing a process in which the angle value calculation part 422 determines the angle value to be notified to the determination part 423. The angle value calculation part 422 calculates the angle value of the driver of the vehicle S on the basis of the captured image captured by the imaging device 1 (step S10). The angle value calculation part 422 acquires the determination result of whether or not the driver is allowed to be in the state of being inattentive to the road ahead, from the state identification part 421 (step S11).

If the state identified by the state identification part 421 is a state where the driver is allowed to be in the state of being inattentive to the road ahead (YES in step S12), the angle value calculation part 422 notifies the determination part 423 about the value at which the difference between the calculated angle value and the reference angle value is less than the threshold value, as the angle value (step S13). On the other hand, if the state identified by the state identification part 421 is a state where the driver is not allowed to be in the state of being inattentive to the road ahead (NO in step S12), the angle value calculation part 422 notifies the determination part 423 about the angle value calculated on the basis of the captured image captured by the imaging device 1 (step S14).

If the difference between the angle value acquired from the angle value calculation part 422 and the reference angle value is equal to or greater than the threshold value, the determination part 423 determines whether or not the driver is in the state of being inattentive to the road ahead. If the difference between the angle value acquired from the angle value calculation part 422 and the reference angle value is equal to or greater than the threshold value, the determination part 423 determines that the driver of the vehicle S is in the state of being inattentive to the road ahead. On the other hand, if the difference between the angle value acquired from the angle value calculation part 422 and the reference angle value is less than the threshold value, the determination part 423 determines that the driver of the vehicle S is not in the state of being inattentive to the road ahead.

If the determination part 423 determines that the driver of the vehicle S is in the state of being inattentive to the road ahead, the determination part 423 notifies the warning part 424 that the driver is in the state of being inattentive to the road ahead. When the warning part 424 receives a notification from the determination part 423 that the driver is in the state of being inattentive to the road ahead, the warning part 424 notifies the alarm device 3 to give a warning.

### <Effect of Safe Driving Determination Apparatus 4>

As described above, the controller 42 includes the state identification part 421 that identifies the state of the vehicle S, and the angle value calculation part 422 that calculates the angle value of the face direction of the driver of the vehicle S. If the state identified by the state identification part 421 is a state where the driver is allowed to be in a state of being inattentive to the road ahead, the angle value calculation part 422 notifies the determination part 423 about i) the calculated angle value or ii) the value in which the difference between the angle value and the reference angle value is less than the threshold value, as the angle value of the driver. The determination part 423 determines whether or not the driver is in the state of being inattentive to the road ahead on the basis of the acquired angle value of the driver.

Therefore, the alarm device 3 does not issue an alarm since the determination part 423 does not determine that the driver is in the state of being inattentive to the road ahead if the driver is allowed to be inattentive to the road ahead, such as when the driver is driving on the curved road. Accordingly, the safe driving determination apparatus 4 can prevent or reduce the issuance of the alarm due to an erroneous determination that the driver is inattentive to the road ahead when the driver does not need to be attentive to the road ahead.

The present disclosure is explained on the basis of the exemplary embodiments. The technical scope of the present disclosure is not limited to the scope explained in the above embodiments and it is possible to make various changes and modifications within the scope of the disclosure. For example, all or part the apparatus can be configured with any unit which is functionally or physically dispersed or integrated. Further, new exemplary embodiments generated by arbitrary combinations of them are included in the exemplary embodiments of the present disclosure. Further, effects of the new exemplary embodiments brought by the combinations also have the effects of the original exemplary embodiments.

### DESCRIPTION OF SYMBOLS

1 imaging device
2 state information detection device
3 alarm device
4 safe driving determination apparatus
41 storage
42 controller
421 state identification part
422 angle value calculation part
323 determination part
324 warning part

1. A safe driving determination apparatus for acquiring an angle value indicating a face direction angle of a driver by using a traveling direction of a vehicle as a reference after capturing an image of the driver of the vehicle with an imaging device to determine whether or not the driver is in a state of being inattentive to the road ahead on the basis of the acquired angle value, the apparatus comprising:
a state identification part that identifies a state of the vehicle;
an angle value identification part that identifies an angle value indicating a face direction angle of at least one of i) a face direction angle of the driver in a yaw direction with respect to the traveling direction or ii) a face direction angle of the driver in a pitch direction with respect to the traveling direction; and
a determination part that determines whether or not the driver is in a state of being inattentive to the road ahead if a difference between the angle value and a reference angle value is equal to or greater than a threshold value, wherein
the angle value calculation part inputs the angle value calculated to the determination part if the state identified by the state identification part is in a state where the driver is not allowed to be in a state of being inattentive to the road ahead, and inputs a value at which the difference from the reference angle value is less than the threshold value, as the angle value to the determination part if the state identified by the state identification part is in a state where the driver is allowed to be in a state of being inattentive to the road ahead.

2. The safe driving determination apparatus according to claim 1, wherein
the state identification part identifies a state of the vehicle as a state where the driver is allowed to be in a state of being inattentive to the road ahead if the vehicle is stopped or if the vehicle is decelerating and the speed of the vehicle is equal to or lower than a threshold speed.

3. The safe driving determination apparatus according to claim 1, wherein
the state identification part identifies a state of the vehicle as a state where the driver is allowed to be in a state of being inattentive to the road ahead if the vehicle is putting on a blinker or if the vehicle is traveling on a curved road.

4. The safe driving determination apparatus according to claim 1, wherein
the state identification part identifies that a state of the vehicle is a state where the driver is not allowed to be in a state of being inattentive to the road ahead if a blinker is on even after a threshold time has elapsed since the vehicle starts to putting on a blinker.

5. The safe driving determination apparatus according to claim 1, wherein
the determination part determines that the driver is in a state of being inattentive to the road ahead if a difference between the angle value and the reference angle value is equal to or greater than a threshold value, and determines that the driver is not in a state of being inattentive to the road ahead if a difference between the angle value and the reference angle value is less than the threshold value.
